(19) United States
(12) Patent Application Publication
(10) Pub. No.: US 2013/0319413 A1
(43) Pub. Date: Dec. 5, 2013

(54) REDUCING SOUND LEVEL IN A RESPIRATORY GAS DELIVERY SYSTEM

(71) Applicants: Eric Porteous, Corona, CA (US); Khalid Said Mansour, Corona, CA (US); Brian W. Pierro, Yorba Linda, CA (US); Geoff Wise, San Jose, CA (US); Christopher M. Varga, Laguna Hills, CA (US)

(72) Inventors: Eric Porteous, Corona, CA (US); Khalid Said Mansour, Corona, CA (US); Brian W. Pierro, Yorba Linda, CA (US); Geoff Wise, San Jose, CA (US); Christopher M. Varga, Laguna Hills, CA (US)

(21) Appl. No.: 13/489,268

(22) Filed: Jun. 5, 2012

(51) Int. Cl. A61M 16/00

(52) U.S. Cl. 128/204.18

(57) ABSTRACT

A system for reducing sound level in a respiratory gas delivery system is described. The system includes an exhaust portion and a sound reducing component that is coupled with the exhaust portion and is configured for absorbing sound. The sound reducing component includes a pathway through which air from within the exhaust portion may move.

### FIELD OF THE INVENTION

The present technology relates generally to the respiratory field. More particularly, the present technology relates to respiratory gas delivery systems.

### BACKGROUND

In the field of respiratory therapy, it is known to provide a continuous positive airway pressure (CPAP) system and method for delivering continuous positive airway pressure, via the nasal cannula, to persons and some instances, to infants. This is particularly true in the case of prematurely born infants who frequently suffer with increased work of breathing due to immature lungs that have the propensity to collapse during exhalation and resist expansion during inhalation.

One particular method of treatment involves the use of nasal cannula that fits sealingly into the nares and are connected to a breathing system that generates a continuous flow of air with above atmospheric pressures, commonly referred to as continuous positive airway pressure (CPAP) therapy. The positive pressure is transmitted through the infant's airways and into the lungs, thereby preventing collapse during exhalation and augmenting expansion during inhalation.

There are a wide variety of devices in use for CPAP. The CPAP devices often comprise what is referred to as a generator body, which is essentially a housing forming a chamber that receives air pressure from tubing. The generator body typically has an exhalation port for air to escape during the exhalation phase, through exhalation tubing. Further, the generator body has a pair of nasal prongs which fit into the patient's nares to supply pressure into the nares.

### DESCRIPTION OF DRAWINGS

FIG. 1 shows a system for reducing sound level in a respiratory gas delivery system, in accordance with embodiments.

FIG. 2 shows a block diagram of a system for reducing sound level in a respiratory gas delivery system, in accordance with embodiments.

FIG. 3A shows a block diagram of a system for reducing sound level in a respiratory gas delivery system, in accordance with embodiments.

FIG. 3B shows a foam segment, in accordance with embodiments.

FIG. 4 shows a flow diagram of an example method for manufacturing a system for reducing sound level in a respiratory gas delivery system, in accordance with embodiments.

FIG. 5 shows a front perspective view of a patient breathing through a respiratory mask through the upper airways.

FIG. 6 shows a patient breathing with an endotracheal tube, wherein the patient's upper airways are bypassed.

FIG. 7 shows a flow diagram of a flow of gas during single limb ventilation.

FIG. 8 shows a flow diagram of a flow of gas during dual limb ventilation.

The drawings referred to in this description should not be understood as being drawn to scale unless specifically noted.

### DESCRIPTION OF EMBODIMENTS

Reference will now be made in detail to various embodiments, examples of which are illustrated in the accompanying drawings. While the subject matter will be described in conjunction with these embodiments, it will be understood that they are not intended to limit the subject matter to these embodiments. On the contrary, the subject matter described herein is intended to cover alternatives, modifications and equivalents, which may be included within the spirit and scope. Furthermore, in the following description, numerous specific details are set forth in order to provide a thorough understanding of the subject matter. However, some embodiments may be practiced without these specific details. In other instances, well-known structures and components have not been described in detail as not to unnecessarily obscure aspects of the subject matter.

### Overview of Discussion

Herein, various embodiments of a system for reducing sound level in a respiratory gas delivery system are described. The description begins with a brief general discussion of traditional respiratory gas delivery systems. This general discussion provides a framework of understanding for a more particularized description which follows, focusing on particular features and concepts of operation associated with one or more embodiments of the described system for reducing sound level.

### Respiratory Gas Delivery Systems

Traditional respiratory gas delivery systems for use in critical care and patient care settings typically involve a generator body, which is essentially a housing forming a chamber that receives air pressure from the tubing of a breathing circuit. The generator body typically has an exhalation port for air to escape during the exhalation phase, through exhalation tubing. Further, the generator body has a pair of nasal prongs which fit into the patient's nares to supply pressure into the nares.

Presently, the sound from the jets that are driven by the generator moves at least through the exhaust tubing, creating a significantly large amount of noise that is potentially harmful to the patient. For example, the high level of noise over extended periods may damage an infant's hearing or interrupt the sleep cycle, requiring it to expend greater energy which could otherwise be used for growth or development. The traditional device that is coupled with the respiratory gas delivery system and is used to reduce the sound level is cumbersome, heavy, and/or at least partially occludes the breathing circuit's air pathway. Further, the traditional respiratory gas delivery system that includes a traditional device for reducing sound level encourages puddling within the air flow path, such that a patient's work of breathing increases during exhalation in order to push against the liquid buildup.

### Reducing Sound Level in a Respiratory Gas Delivery System

As will be described in detail below, embodiments provide a system for reducing sound level in a respiratory gas delivery system and a method for manufacturing the system. For example, in one embodiment, the system includes an exhaust tubing and a foam segment coupled with the exhaust tubing. The foam segment absorbs sound as well as includes a pathway through which air from within the exhaust tubing may move.

Embodiments provide many benefits over traditional systems. For example, embodiments enable the sound level within the exhaust tubing of a respiratory gas delivery system (e.g., single-limb respiratory circuit [e.g., nCPAP]) to be reduced significantly. Embodiments also allow for this sound-reducing foam segment to be incorporated within the respiratory gas delivery system without occluding the air pathway through the exhaust tubing, since the foam segment itself provides for an air pathway. Additionally, the foam segment maintains its effectiveness in reducing the sound level within the exhaust tubing, regardless of its location within the exhaust tubing. Further, embodiments are smaller, lighter, more inexpensive, and more robust than traditional devices used to reduce the sound level within a respiratory gas delivery system. Moreover, embodiments discourage liquid from puddling, thereby decreasing the patient's work of breathing during exhalation against this liquid buildup.

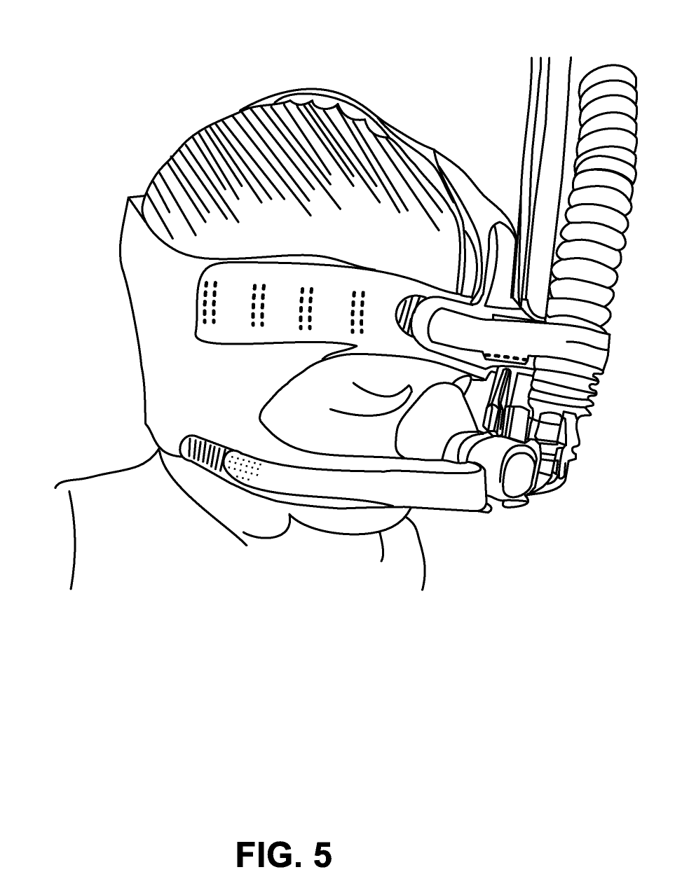

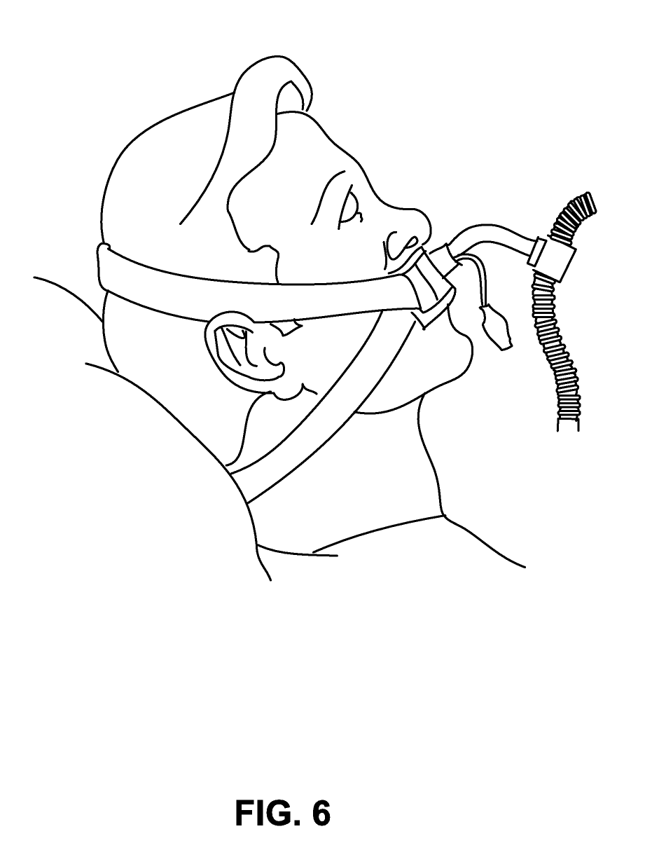

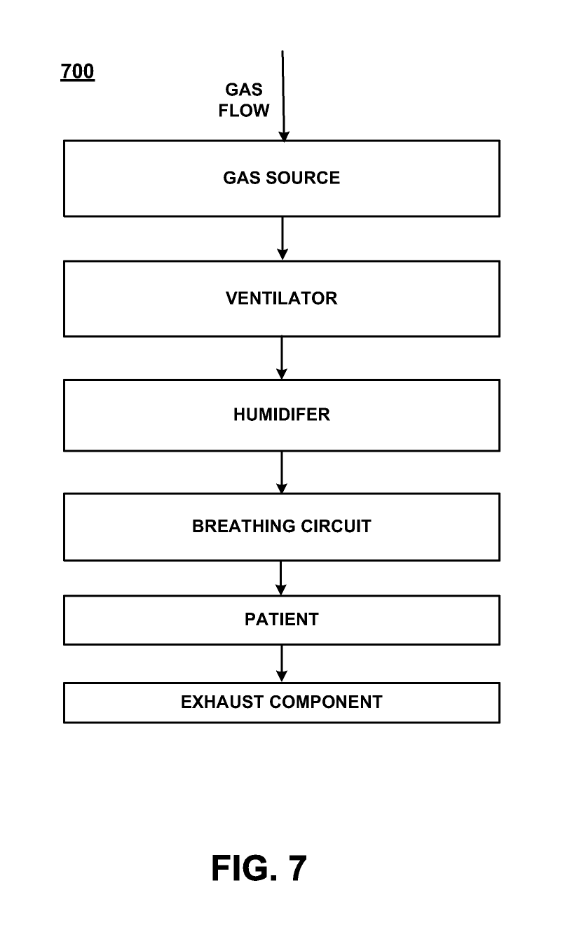

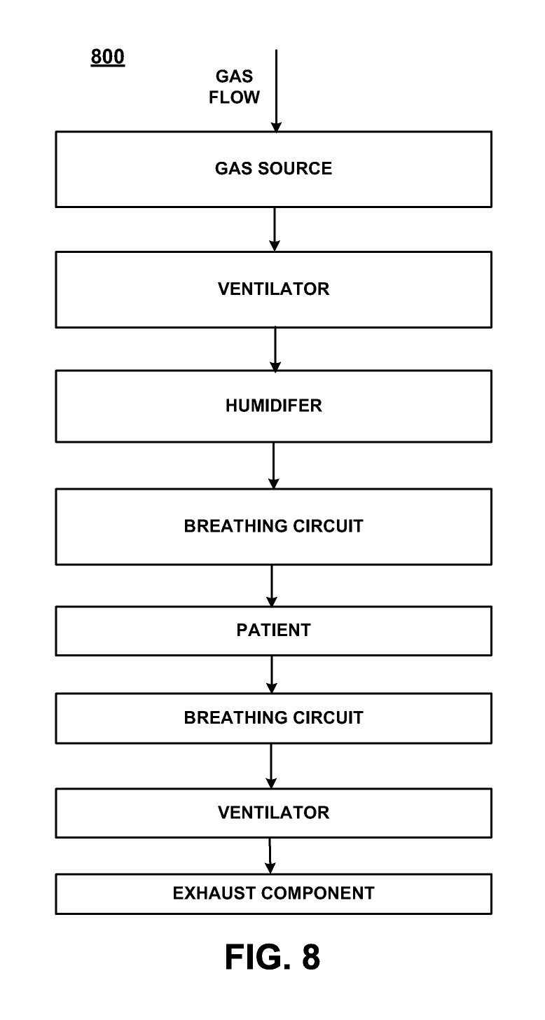

It should be noted that the methods and devices described herein may be used in various modes of respiratory care, including, but not limited to, non-invasive single limb ventilation, dual-limb invasive ventilation, dual-limb non-invasive ventilation, continuous positive airway pressure (CPAP), bubble CPAP, bi-level positive airway pressure (BiPAP), intermittent positive pressure (IPPB), bland aerosol therapy and oxygen therapy. In general, non-invasive single and dual-limb ventilation refers to the delivery of ventilator support using a mechanical ventilator, with one or multiple limbs, connected to a mask or mouthpiece instead of an endotracheal tube or tracheostomy interface. For example, FIG. 5 shows a front perspective view of a patient breathing with a mask through the upper airways (using a non-invasive ventilation system). A dual-limb invasive therapy refers to the delivery of ventilator support using a mechanical ventilator, with multiple limbs, connected to an endotracheal tube. For example, FIG. 6 illustrates a patient breathing with an endotracheal tube, wherein the patient's upper airways are bypassed (using an invasive ventilation system). Further, FIGS. 7 and 8 illustrate flow diagrams 700 and 800, respectively, of the flow of gas during single limb and dual limb ventilation, respectively. More particular, 700 of FIG. 7, with regards to single limb ventilation, shows gas flowing from a gas source to a ventilator, to a humidifier, to a breathing circuit, to a patient, to an exhaust component. In contrast, 800 of FIG. 8, with regards to dual limb ventilation, shows gas flowing from a gas source to a ventilator, to a humidifier, to a breathing circuit, to a patient, to a breathing circuit, to a ventilator, to an exhaust component.

CPAP refers to the maintenance of positive pressure in the airway throughout a respiratory cycle. Bubble CPAP refers to a procedure that doctors use to help promote breathing in premature newborns. In bubble CPAP, positive airway pressure is maintained by placing the expiratory limb of the circuit under water. The production of bubbles under the water produces a slight oscillation in the pressure waveform. BiPAP refers to the maintenance of a baseline positive pressure during inspiration and expiration, but with brief increases of this pressure periodically. IPPB refers to the non-continuous application of positive airway pressure when, for example, an episode of apnea is sensed. Bland aerosol therapy refers to the delivery of hypotonic, hypertonic, or isotonic saline, or sterile water in aerosolized form, to a patient as a medical intervention. Oxygen therapy refers to the delivery of oxygen to a patient, as a medical intervention.

The following discussion describes the architecture and operation of embodiments.

Breathing circuits are utilized to deliver such medical support as air and anesthetics from a machine that creates an artificial environment to a patient via tubes. Breathing circuits are used in surgical procedures, respiratory support and respiratory therapies. For example, in a most general case, breathing circuits include an inspiratory limb running from a ventilator to a patient and an expiratory limb running from the patient back to the ventilator.

The ventilator pushes gas through the inspiratory limb to reach the patient. The patient inhales this pushed gas and exhales gas into the expiratory limb. For purposes of the embodiments, any portion of the breathing circuit could be considered a patient circuit or conduit. It should be appreciated that embodiments are well suited to be used in any portion of the expiratory limb of the patient circuit. For purposes of the embodiments described herein, the expiratory limb is considered a whole or a subset of an exhaust portion of the respiratory gas delivery system. The exhaust portion is any part of the respiratory gas delivery system through which exhaled gas moves.

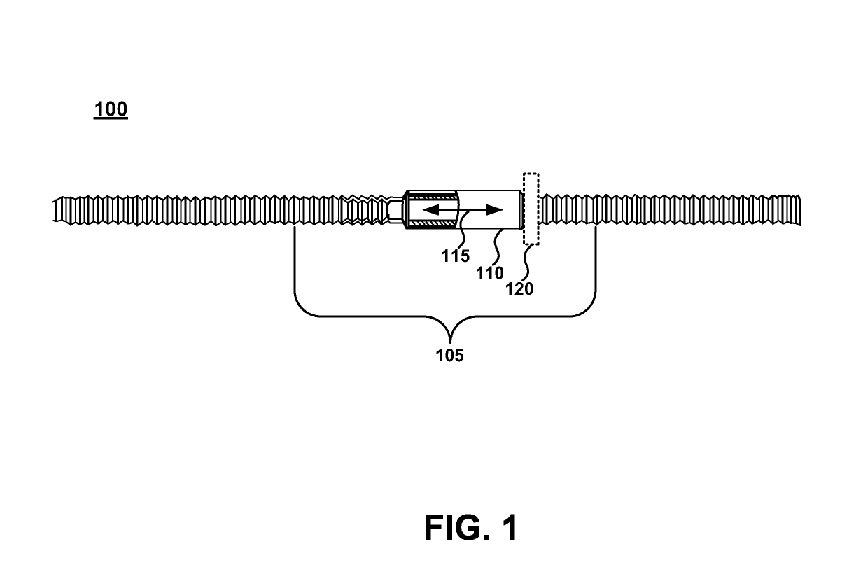

FIG. 1 shows a system 100 for reducing sound level in a respiratory gas delivery system. The system includes an exhaust portion 105 and a sound reducing component 110 coupled with the exhaust portion 105. The sound reducing component 110 absorbs sound and includes a pathway 115 through which air from within the exhaust portion 105 may move.

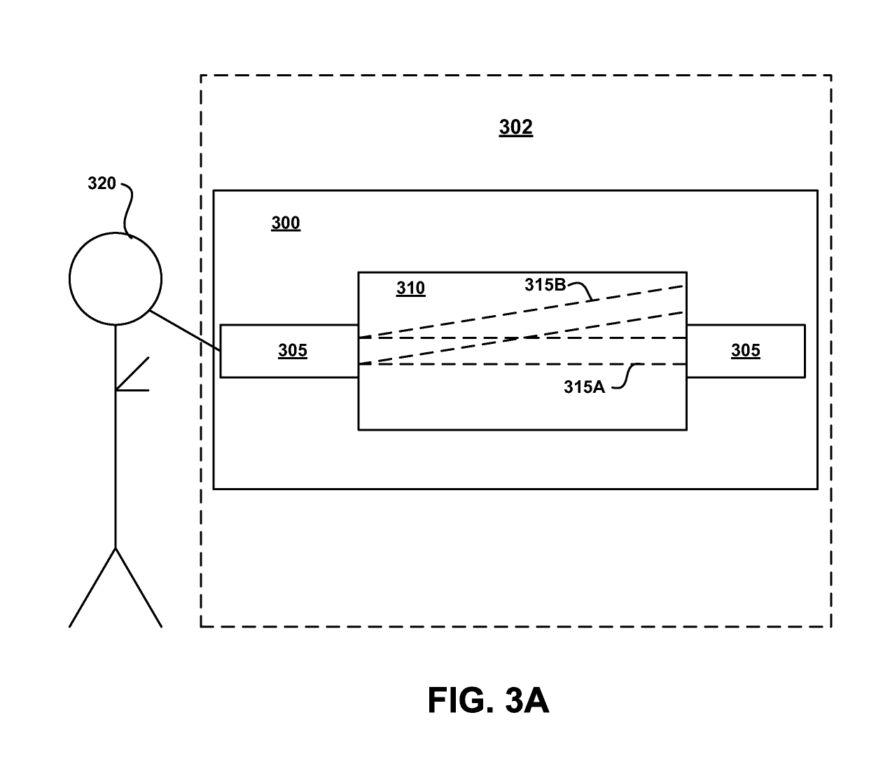

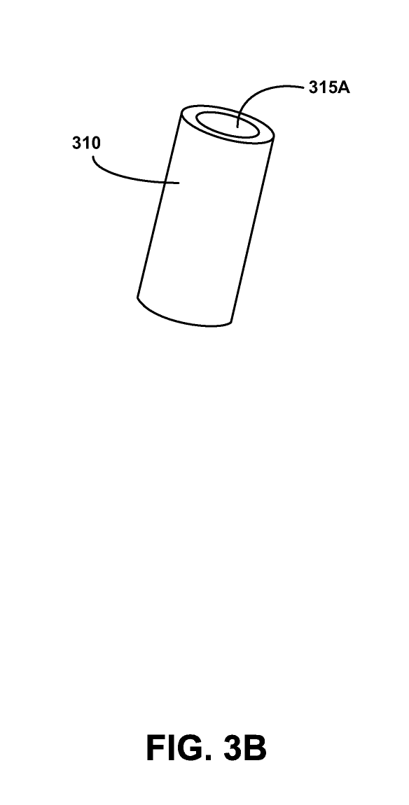

In various embodiments the sound reducing component 110 may be at least one of the following: a foam segment (described below in association with FIGS. 3A and 3B); a spray coating; an open cell material providing the pathway 115 through which air may travel; a closed cell material; polyurethane; an entirety of the exhaust portion 105; and a wicking component.

It should be noted that the sound reducing component 110 may be of any size facilitating a reduction in sound level and located at any portion of the entire length of the exhaust portion 105, as well as being sized to run along the entire length of the exhaust portion 105. The sound reducing component 110 may be of any length that retains the characteristics of being capable of reducing sound level within the respiratory gas delivery system 100.

In yet other embodiments, the sound reducing component 110 may be of any material or combination of materials that enable air to pass there though (via either a hole in the middle of the material or an open cell material) while also having properties that reduce the sound level.

Further, in various embodiments, the sound reducing component 110 may be disposable and capable of wicking a liquid away from a first location within the respiratory gas delivery system 100. This wicking capability and/or the open cell material characteristic enables rainout to escape a location and/or be absorbed.

In yet other embodiments, the sound reducing component 110 may be of any material or combination of materials that enable air to pass there though (via either a hole in the middle of the material or an open cell material) while also having properties that are capable of reducing the sound level in the respiratory gas delivery system 100.

Additionally, in one embodiment, the sound reducing component 110 is a muffler shape, conforming to the inner surface of the exhaust portion 105. However, it should be noted that the sound reducing component 110 may be of any shape that serves to absorb the sound traveling through the exhaust portion 105, while also allowing air to travel through it.

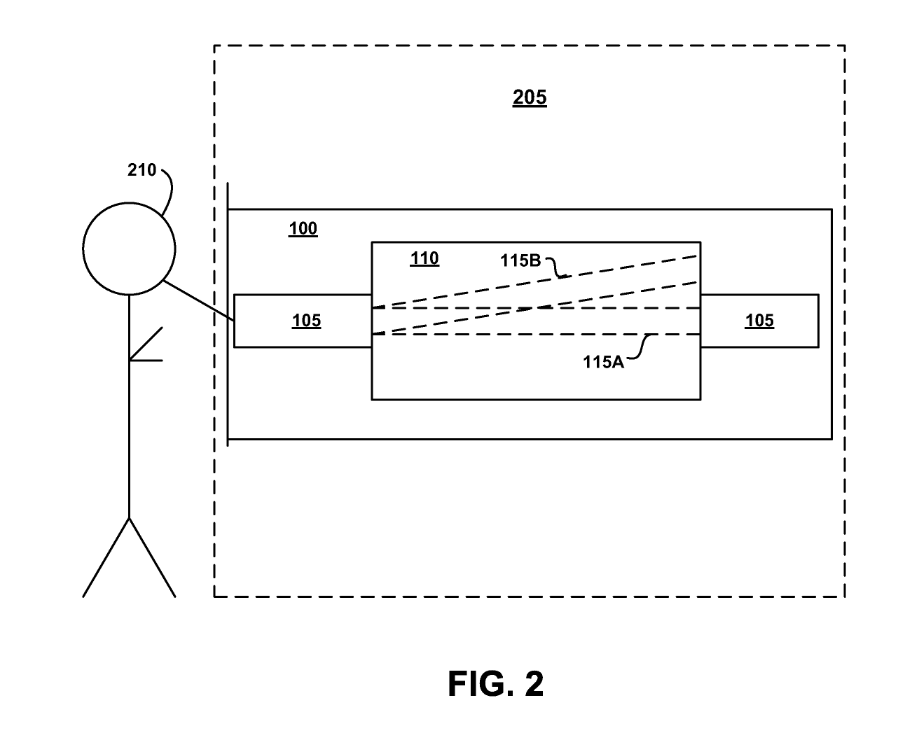

FIG. 2 shows a system 100 for reducing sound level in a respiratory gas delivery system 205, in accordance with embodiments. System 100 is coupled with the patient 210. As shown, in one embodiment, a pathway 115 (of FIG. 1) within the sound reducing component 110, such as pathway 115A, is directed towards an interior of the exhaust portion 105. In another embodiment, a pathway 115, such as pathway 115B, is directed towards an environment outside of the exhaust portion 105. For example, the exhaust air may go through pathway 115B that directs the exhaust air outside of the system 100, and ultimately outside of the environment of the respiratory gas delivery system 205.

In one embodiment, the respiratory gas delivery system 205 is an infant respiratory gas delivery system. In another embodiment, the respiratory gas delivery system 205 is a CPAP system. In one embodiment, the CPAP system is an infant nasal CPAP (nCPAP) system.

In one embodiment, the reduction in the sound level results in at least one decibel of sound reduction.

FIG. 3A shows a system 300 for reducing sound level in a respiratory gas delivery system 302, in accordance with embodiments. The system 300 is coupled with the patient 320. In one embodiment, a foam segment 310 is coupled with an exhaust tubing 305. The foam segment 310 absorbs sound. The foam segment 310 includes, alternatively, pathways 315A or 315B, through which air from within the exhaust tubing 305 may travel. The pathways 315A and 315B perform the same functions as pathways 115A and 115B, respectively, of FIG. 2. In one embodiment, the foam segment 310 includes a ridged surface such that air may flow around the foam segment 310, through the exhaust tubing 305, instead of through the foam segment 310. It should be appreciated that the foam segment 310 is the sound reducing component 110 of FIG. 1, in one embodiment. Additionally, the exhaust tubing 305, in one embodiment, is the exhaust portion 105 of FIGS. 1 and 2.

FIG. 3B shows an enlarged foam segment 310 with pathway 315A of FIG. 3A, in accordance with embodiments. It should be appreciated that the pathways 315A and 315B (as well as the pathways 115A and 115B) may be of any diameter that enables a sufficient amount of air to flow there through without causing a patient's breathing work rate to increase.

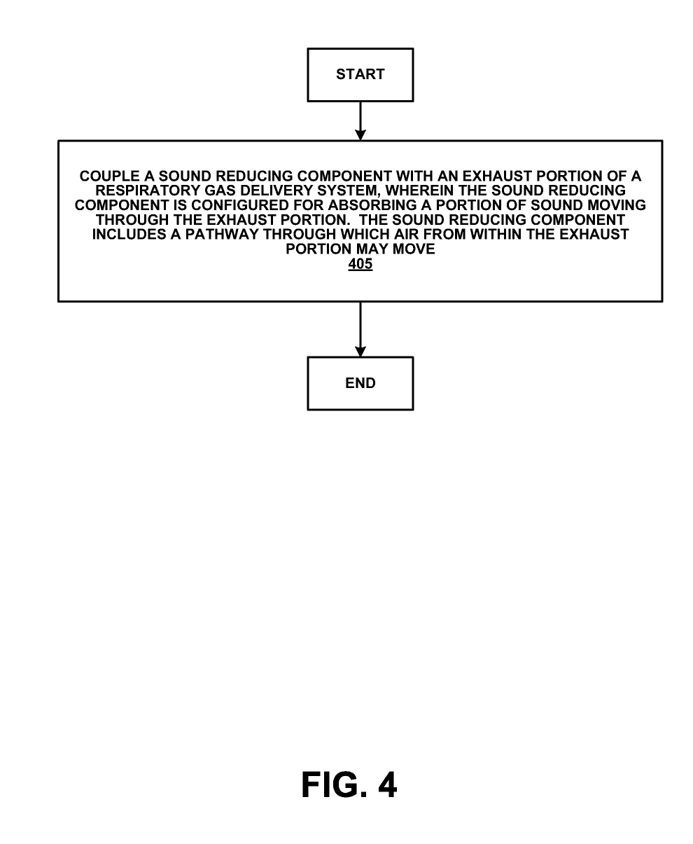

FIG. 4 shows a flow chart 400 for manufacturing a system for reducing sound level in a respiratory gas delivery system, in accordance with embodiments. With reference to FIGS. 1-4, in one embodiment, at step 405, the sound reducing component 110 is coupled with an exhaust portion 105 of the respiratory gas delivery system 205, wherein the sound reducing component 110 absorbs a portion of the sound moving through the exhaust portion 105. In one embodiment, the sound reducing component 110 is snapped into the exhaust portion 105. The sound reducing component 110 remains within the exhaust portion 105 through a stabilizing means, such as, but not limited to, an interference fit, or through at least one connecting component attaching the sound reducing component 110 to the exhaust portion 105 (e.g., see connector 120 of FIG. 1 as an example).

In one embodiment, and as described herein, during manufacturing, a foam segment 310 and/or a spray coating is coupled with the exhaust portion 105. In various embodiments, and as described herein, a sound reducing component 110, that is an open cell material providing a pathway through which the air may move, is coupled with the exhaust portion 105. In yet other embodiments, the sound reducing component 110 that is coupled with the exhaust portion 105 includes a pathway 115 (including pathways 115A and 115B) through which air from within the exhaust portion 105 may move, wherein the pathway 115 is directed to an interior of the exhaust portion 105 and/or an environment outside of the exhaust portion 105.

In various embodiments, and as described herein, during manufacturing the sound reducing component 110 is coupled with an exhaust portion 105 of an infant respiratory gas delivery system or a CPAP system. In one embodiment, the CPAP system is an nCPAP system.

All statements herein reciting principles, aspects, and embodiments of the present technology as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present technology, therefore, is not intended to be limited to the embodiments shown and described herein. Rather, the scope and spirit of present technology is embodied by the appended claims.

What is claimed is:

1. A system for reducing sound level in a respiratory gas delivery system, said system comprising:
an exhaust portion; and
a sound reducing component coupled with said exhaust portion and configured for absorbing sound, said sound reducing component comprising:
a pathway through which air from within said exhaust portion may move.

2. The system of claim 1, wherein said sound reducing component comprises:
a foam segment.

3. The system of claim 1, wherein said sound reducing component comprises:
a spray coating.

4. The system of claim 1, wherein said sound reducing component comprising:
an open cell material providing said pathway through which air may travel.

5. The system of claim 1, wherein said sound reducing component comprises:
a closed cell material.

6. The system of claim 1, wherein said sound reducing component comprises:
polyurethane.

7. The system of claim 1, wherein said sound reducing component comprises:
an entirety of said exhaust portion.

8. The system of claim 1, wherein said sound reducing component comprises:
a wicking component.

9. The system of claim 1, wherein said pathway is directed to an interior of said exhaust portion.

10. The system of claim 1, wherein said pathway is directed to an environment outside of said exhaust portion.

11. The system of claim 1, wherein said respiratory gas delivery system comprises:
an infant respiratory gas delivery system.

12. The system of claim 1, wherein said respiratory gas delivery system comprises:
a continuous positive airway pressure (CPAP) system.

13. The system of claim 12, wherein said CPAP system comprises:
an infant nasal CPAP (nCPAP) system.

14. The system of claim 1, wherein a reduction in a sound level results in at least 1 decibel of sound reduction.

15. A system for reducing sound level in a respiratory gas delivery system, said system comprising:
an exhaust tubing; and
a foam segment coupled with said exhaust tubing and configured for absorbing sound, said foam segment comprising:
a pathway through which air from within said exhaust tubing may travel.

16. A method for manufacturing a system for reducing sound level in a respiratory gas delivery system, said method comprising:
coupling a sound reducing component with an exhaust portion of said respiratory gas delivery system, wherein said sound reducing component is configured for absorbing a portion of sound moving through said exhaust portion and comprises:
a pathway through which air from within said exhaust portion may move.

17. The method of claim 16, wherein said coupling said sound reducing component comprises:
coupling a foam segment with said exhaust portion.

18. The method of claim 16, wherein said coupling said sound reducing component comprises:
coupling a spray coating with said exhaust portion.

19. The method of claim 16, wherein said coupling said sound reducing component comprises:
coupling an open cell material providing said pathway though which said air may move.

20. The method of claim 16, wherein said coupling said sound reducing component comprises:
coupling said sound reducing component that comprises a pathway through which air from within said exhaust portion may move, wherein said pathway is directed to an interior of said exhaust portion.

21. The method of claim 16, wherein said coupling said sound reducing component comprises:
coupling said sound reducing component that comprises a pathway through which air from within said exhaust portion may move, wherein said pathway is directed to an environment outside of said exhaust portion.

22. The method of claim 16, wherein said coupling said sound reducing component comprises:
coupling said sound reducing component with said exhaust portion of an infant respiratory gas delivery system.

23. The method of claim 16, wherein said coupling said sound reducing component comprises:
coupling said sound reducing component with said exhaust portion of a continuous positive airway pressure (CPAP) system.

24. The method of claim 23, wherein said coupling said sound reducing component comprises:
coupling said sound reducing component with said exhaust portion of an infant nasal CPAP (nCPAP) system.
